(19) United States
(12) Patent Application Publication
(10) Pub. No.: US 2011/0306131 A1
(43) Pub. Date: Dec. 15, 2011

(54) INDUCTION OF PLURIPOTENT STEM CELLS INTO MESODERMAL LINEAGES

(76) Inventor: Diane Elizabeth Rudy-Reil, Plymouth, WI (US)

(21) Appl. No.: 13/215,202

(22) Filed: Aug. 22, 2011

Related U.S. Application Data
(63) Continuation of application No. 11/876,769, filed on Oct. 22, 2007.
Application No. 11/876,769 is a continuation-in-part of application No. 11/159,567, filed on Jun. 22, 2005, now abandoned.
(60) Provisional application No. 60/581,946, filed on Jun. 22, 2004.

(51) Int. Cl. C12N 5/071

(52) U.S. Cl. 435/366

(57) ABSTRACT

The present invention provides a method of inducing mesoderm derived cells from pluripotent stem cells. In contrast to methods known in the art that are often designed to replicate in vivo events of mesoderm induction, the present invention provides a unique, yet simple, method whereby pluripotent stem cells are mesodermally primed in the presence of factors that concomitantly inhibit the spontaneous differentiation of endoderm and ectoderm during expansion and suspension steps. Exposure and/or adherence of primed aggregates to a extracellular matrix that promotes the commitment and survival of induced mesoderm progenitors, followed by exposure to various mesoderm associated factors, allows for the subsequent induction of such cells into terminally differentiated lineages, such as cardiomyocytes. End products of this induction system will ultimately provide an unlimited source of mesoderm-derived cell types for therapeutic and pharmacological purposes.

### CROSS-REFERENCE TO RELATED APPLICATIONS

This application is a continuation of application Ser. No. 11/876,769, filed on Oct. 22, 2007, which is a continuation-in-part of application Ser, No. 11/159,567, filed on Jun. 22, 2005, now abandoned. Application Ser. No. 11/159,567 is a provisional conversion of application No. 60/581,946, filed on Jun. 22, 2004. These applications are herein incorporated by reference.

### BACKGROUND OF THE INVENTION

In 1998, Thomson et al. determined that undifferentiated embryonic stem cells can be isolated from human blastocysts and cultured indefinitely without losing their ability to differentiate into cell types consistent with naturally-occurring events of human development.

Similarly, Doetschmann et al. (1985) observed that if isolated embryonic stem cells are placed into suspension, they will spontaneously form aggregates termed embryoid bodies (EBs) within which cells from all three germ layers can be identified. Due to the high incidence of non-mesodermal cell types, however, the percentage of spontaneously differentiated mesoderm lineages such as cardiac myocytes, derived from such methods is unacceptably low.

Although possible to separate mesoderm lineages from the mixture of cells containing the endoderm, ectoderm and mesoderm derived cells, these methods require additional costly and inefficient separation and enrichment steps. Accordingly, a method of directly inducing a homogenous population of mesoderm derived cells exists.

### SUMMARY OF THE INVENTION

The present invention provides a method of inducing mesoderm derived cells from pluripotent stem cells. In contrast to methods known in the art that are often designed to replicate in vivo events of mesoderm induction, the present invention provides a unique, yet simple, method whereby pluripotent stem cells are mesodermally primed in the presence of factors that concomitantly inhibit the spontaneous differentiation of endoderm and ectoderm during expansion and suspension steps. Exposure and/or adherence of primed aggregates to a extracellular matrix that promotes the commitment and survival of induced mesoderm progenitors, followed by exposure to various mesoderm associated factors, allows for the subsequent induction of such cells into terminally differentiated lineages, such as cardiomyocytes. End products of this induction system will ultimately provide an unlimited source of mesoderm-derived cell types for therapeutic and pharmacological purposes.

Other features and advantages of the present invention will become apparent after study of the specification and claims that follow.

### Definitions

As used herein, “pluripotent stem cells” and “stem cell” refer to human embryonic stem cells and human pluripotent stem cells derived from non-embryonic stem cells. Although human pluripotent stem cells are preferred, the method is also applicable to non-human pluripotent stem cells, such as primate and murine.

As used herein, “fibroblast layer” refers to a feeder layer such as a mouse embryonic fibroblast (MEF) feeder layer, human embryonic fibroblast (HEF) feeder layer, or any alternative fibroblast lines used to support cell growth during cell expansion.

The term “mesodermal priming” refers to the concomitant exposure of pluripotent stem cells to a “priming medium” during both the expansion and suspension steps of mesodermal progenitor induction.

As used herein, “priming medium” refers to a medium that contains at least one factor, such as basic Fibroblast Growth Factor (bFGF), that promotes the induction activity of mesoderm and at least one factor that inhibits the activity of factors required for endoderm and ectoderm differentiation. In the preferred priming medium, bFGF serves to both promote the induction of mesoderm and to inhibit the activity of Bone Morphogenetic Protein (BMP), which is a factor that promotes endoderm and ectoderm differentiation. In addition, the priming medium may also include mouse embryonic fibroblast conditioned medium (MEF-CM), human embryonic fibroblast conditioned medium (HEF-CM), medium conditioned by other or alternative fibroblast lines, and other Fibroblast Growth Factor (FGF) isoforms.

As used herein, “substrate” refers to a compound, preferably fibronectin that promotes the commitment and survival of the induced mesodermal progenitor cells.

As used herein, “target mesoderm derived cells” refers to cardiomyocytes, endothelial and smooth muscle cells, mesodermal mesenchyme, hematopoietic cells, skeletal muscle, adipocytes, chondrocytes, osteocytes, and other cells that can be induced from mesodermal progenitor cells.

As used herein, “Mesoderm Associated Factors,” or “MesA factors,” refers to compounds, used alone or in combination, capable of differentiating mesodermal progenitor cells into target mesoderm derived cells. MesA factors include, but are a not limited to, mesoderm explants, mesoderm conditioned medium, mesoderm secreted growth factors, cytokines, and synthetic equivalents of the same. MesA factors for cardiogenesis include but are not limited to, mesoderm explants, mesoderm conditioned medium or mesoderm secreted growth factors such as Hepatocyte Growth Factor (HGF) and its isoforms, FGF and its isoforms and Epidermal Growth Factor (EGF). MesA factors for vasculogenesis include, but are not limited to mesoderm derived BMP and endothelium promoters.

All publications and patents mentioned in this application are herein incorporated by reference for any purpose.

### DETAILED DESCRIPTION OF THE INVENTION

The present invention provides a method of inducing relatively pure populations of target mesoderm derived cells from pluripotent stem cells without the need for separation and enrichment steps. Unlike methods of mesoderm differentiation that result in the differentiation of endoderm and ectoderm as well as mesoderm, the method of the present invention obtains homogenous populations of mesoderm derived cells by first, inducing mesodermal progenitor cells from pluripotent stem cells, and then inducing target mesoderm derived cells from the mesodermal progenitor cells.

Induction of Mesodermal Progenitor Cells

To promote the induction of mesodermal progenitor cells, pluripotent stem cells are transferred to a fibroblast layer and exposed to a priming medium to initiate mesodermal priming.

The mesodermal priming continues as the stem cells expand in the presence of the priming medium until they have established cell to cell contacts, preferably to a confluence of at least 60%.

The expanded and primed stem cells are then dissociated from the fibroblast layer with collagenase or equivalent material. The dissociated cells are then placed into suspension with the priming medium and allowed to aggregate for a predetermined time period. The predetermined time is preferably between 1 and 4 days and most preferably 3 days.

Induction of the mesodermal progenitor cells is complete when the primed stem cell aggregates formed during the suspension step are exposed or adhered to a substrate that promotes commitment and survival of the cells. The substrate is preferably fibronectin.

The continuous exposure of the stem cells to the priming medium throughout the expansion and suspension stages is critical to the mesodermal priming and subsequent mesodermal progenitor cell induction. If the priming medium is not present during the suspension stage as well as the expansion stage, the inhibition of the endoderm and ectoderm promoting factors will be lost. Consequentially, any subsequent differentiation of stem cells suspended in the absence of the priming medium will result in a mixture of endoderm, ectoderm and mesoderm lineages in proportions similar to those found naturally occurring in an embryo.

Induction of Mesodermal Progenitor Cells into Target Mesoderm Derived Cells

Once the induction of the mesodermal progenitor cells is complete, target mesoderm derived cells are induced by exposing the mesodermal progenitor cells to a corresponding MesA factor for a predetermined period of time. The specific induction medium composition and time parameters will vary depending upon the MesA factor selected.

The following non-limiting examples are provided to illustrate the application of the method in regard to selected target mesoderm derived cells. These examples do not represent the full scope of combinations available through the present invention.

Induction of Mesodermal Progenitor Cells into Cardiomyocytes

Mesodermal progenitor cells induced in accordance with the present invention are exposed to at least one MesA factor known to induce cardiomyocyte differentiation for a time period between 7 and 21 days. This group includes, but is not limited to, precardiac explants, precardiac mesoderm conditioned medium and mesoderm secreted growth factors such as HGF and its isoforms, FGF and its isoforms, and EGF and its isoforms.

In a preferred embodiment, cardiomyocytes are induced when the mesodermal progenitor cells are exposed to a combination of MesA factors HGF and bFGF.

Induction of Mesodermal Progenitor Cells into Endothelium

Mesodermal progenitor cells induced in accordance with the present invention are exposed to at least one MesA factor known to induce endothelium differentiation for a time period between 7 to 21 days. This group includes but is not limited to mesodermally active BMP and endothelium promoters, and preferably BMP-4.

### EXAMPLES

Induction of Mesoderm Progenitor Cells

Mesoderm progenitor induction was accomplished by expanding pluripotent stem cells on mitotically-inactive MEF feeder cells (50,000 cells/cm², and prepared 24 hours in advance) in the presence of a priming medium comprising MEF-CM and bFGF. More specifically, the primed medium included 80% DMEM F-12, 20% KO Serum Replacement, 1% NEAA, 1.0 mmol/L L-glutamine, 0.1 mmol/L β-mercaptoethanol that had been previously conditioned by MEFs in accordance with Xu et al. (2001) and 4 ng/ml bFGF. The priming medium was replenished daily. The primed stem cells were grown to a confluence of approximately 60% in the presence of the priming medium.

The expanded and primed stem cells were then dissociated from the MEF feeder cells following a brief (approximately 3 minutes at 37° C.) exposure to collagenase (200 U/mL) and nearly intact colonies of the primed stem cells were placed into suspension in the presence of the priming medium (MEF-CM plus 4 ng/ml bFGF) for a maximum of 3 days. The priming medium was replenished daily. This step was performed in an effort to re-establish and/or increase the level of cell-cell contacts potentially lost during dissociation but required for mesodermal induction. Commitment of the primed stem cells to mesodermal progenitor cells occurred with the subsequent exposure of the primed stem cells onto a fibronectin substrate.

Induction of Mesodermal Progenitor Cell differentiation via MesA Factors

Formation of Precardiac Mesoderm Explant MesA Factor for Murine Embryonic Stem Cell Co-culture and/or Derivation of Mesoderm-Conditioned Medium

At least 1 hour before dissection, 15 -mm-diameter culture wells were incubated with 50 μg/mL fibronectin at room temperature, the excess of which was replaced with 1.0 mL explant medium (DMEM, 10% FBS, 50 μg/mL gentamicin) prior to the addition of explants. Explants of lateral plate mesoderm, microdissected from Hamburger & Hamilton stage 5 chick embryos were used. Briefly, embryos were removed from the yolk and placed in PBS. With ventral side up, intact sheets of mesoderm were gently teased from underlying ectoderm following a 1-minute digestion in ice-cold collagenase/dispase (1 mg/mL) and removal of overlying endoderm. Three or 4 explants were placed in each well and cultured for 24 hours before the addition of primed stem cell aggregates.

Formation of Precardiac Mesoderm-Conditioned Medium MesA Factor

Precardiac Mesoderm-Conditioned Medium was prepared by placing 15-20 precardiac mesoderm explants in 15 -mm-diameter fibronectin-coated wells containing 1.0 mL explant medium and incubating at 37° C. for 48 hrs. Conditioned medium was then transferred to new 15 -mm fibronectin-coated wells, followed immediately by implantation of 4-5 mesoderm progenitor cell aggregates. Cardiac differentiating cells were replenished with mesoderm-conditioned medium daily for 7 days, after which cells were fed with unconditioned explant medium for an additional 1-2 weeks.

Cardiomyocyte Induction of Mesoderm Progenitor Cells via Co-culture with Mesoderm explant MesA Factor

Differentiation of mesoderm progenitor cell aggregates was induced by implanting the mesoderm progenitor cell aggregates into fibronectin-coated culture wells and atop or adjacent to explanted mesoderm tissue. All cultures were maintained in an unconditioned explant medium, which was replenished daily. The day of aggregate plating was designated as Induction Day 0 and cultures were monitored for rhythmic contractility for up to 3 weeks.

Cardiac Induction of Mesoderm Progenitor Cells via Precardiac Mesoderm-conditioned medium MesA Factor

For experiments in which mesoderm explants were replaced with explant-conditioned medium, the latter was prepared as described above and replenished on a daily basis through Induction day 7; after day 7, daily replenishment was made using fresh, unconditioned explant medium. Also, mesoderm progenitor cell aggregates were plated directly onto a fibronectin (50 μg/ml) substrate and monitored for rhythmic contractility for 2-3 weeks.

Mesoderm-derived cardiomyocytes were observed to beat 4-9 days following exposure of the mesoderm progenitor cells to mesoderm explants or mesoderm-conditioned medium. Initially, contractile activity was restricted to tiny areas within the differentiating aggregate; these beating areas rapidly increased in size and number, resulting in non-synchronous beating throughout the aggregate by 12-14 days in culture.

Cardiac Induction of Mesoderm Progenitor Cells via Precardiac Mesoderm-secreted Growth Factors

The potency of precardiac mesoderm conditioned medium to induce cardiogenesis in virtually 100% of Mesoderm Progenitor Cells afforded a unique opportunity to identify heretofore unknown cardiogenic factors secreted by this embryonic tissue. As such, mesoderm conditioned medium was analyzed against a custom-designed cytokine antibody array. Prior to performing these analyses, however, it was necessary to reduce the amount of FBS in this medium from 10% to the minimal amount required for precardiac explant differentiation. This was determined (n=12) by culturing explants in medium supplemented with 0, 2, 4, 6, 8 and 10% FBS for one week and comparing the effects of FBS concentration against explant viability and beating activity. Such efforts surprisingly revealed that growth of precardiac explants was optimal in medium supplemented with 2% FBS.

To identify secreted cardiogenic factors, 1.0 ml of medium (w/2% FBS) was conditioned by 25-30 explants for 48 hrs, then applied directly to a custom-designed array of membrane-bound antibodies specific to growth factors implicated in various phases of cardiac development. Because freezing reduces cardiogenic potency, only fresh explant-conditioned medium was analyzed. Controls consisted of non-conditioned medium w/ and w/o 2% FBS. Subsequent measurements of signal intensities were performed using NIH Image J software.

In an attempt to establish statistical significance, extensive efforts were made to minimize experimental variability between each membrane set; however, a certain level of variation was unavoidable (e.g., due to potential differences in precardiac explant secretions and individual membrane sensitivities). A determination of statistical relevance was also difficult due to the limited number of array analyses that could be performed (n=4). Nonetheless, a comparison of signal intensities between the 36 growth factors tested revealed three putative cardiogenic growth factors, bFGF (FGF-2), HGF and EGF, that were present in conditioned medium at levels averaging 2-8 fold greater than those detected in unconditioned medium w/FBS. (It is important to note that identification of mesoderm secreted growth factors was limited to the number of antibodies available for analyses by the manufacturer. Also, the manufacturer recognizes significant cross-reactivity between isoforms of arrayed proteins; thus, it is likely that specific and/or currently unknown protein isoforms (e.g., embryonic and/or cardiogenic) present in mesoderm-conditioned medium and responsible for cardiac induction were not identified using this technique.)

Based on the outcome of the cytokine membrane analyses, it was of interest to examine the isolated and combined cardiogenic effects of the growth factors identified. To accomplish this, ES cells were expanded and suspended as previously described.

Resultant aggregates were then cultured for 7 days in non-conditioned medium supplemented with 10% FBS and varying concentrations of growth factors for 7 days (note that an extended 14-day treatment did not alter the outcome of results). At Induction Day 8, growth factor medium was replaced with untreated medium and differentiating ES cell-derived aggregates were cultured for an additional week prior to fixation. Cardiogenesis was assessed based upon direct observations of contractile activity, cell morphology and results of immunostaining with antibodies specific to sarcomeric muscle markers. Levels of cardiac induction were also compared with those observed in control cultures without growth factor supplementation.

Cardiac Induction of Mesoderm Progenitor Cells via HGF and HGF+bFGF.

The presence of bFGF (FGF-2) in conditioned medium was consistently observed at levels averaging approximately 8-fold greater than those in non-conditioned DMEM plus FBS. Thus, it was of interest to examine the cardiogenic potency of this growth factor which was accomplished by treating mesoderm progenitor cells with bFGF at concentrations of 5, 50 and 100 ng/ml. bFGF alone was minimally cardiogenic—although 6/13 aggregates treated with 50 ng/ml of this protein exhibited contractile activity after approximately 12 days in culture, immunostaining with antibodies specific to sarcomeric actin revealed that myocyte differentiation was confined to a minute population of cells (<1%).

HGF in mesoderm conditioned medium was consistently and significantly detected at levels approximately 2-fold greater than those in unconditioned medium plus FBS. However, in contrast to observations described above, aggregates treated with HGF alone displayed unique characteristics that were evident by Induction Day 4; for example, differentiating aggregates remained more compact throughout the two-week culture period and maintained a homogeneous appearance despite the early detection of unknown cell types (see Controls and BMP-4 below) in 1/7 aggregates exposed to 5 ng/ml HGF and 1/8 exposed to 50 ng/ml. Only 2/7 aggregates cultured in 50 ng/ml HGF were observed to beat at Induction Day 7; but immunostaining revealed that 100% of cultures exposed to 50 ng/ml HGF for 7 days contained large cardiogenic regions that, together, approximated >50% of total cells. The effects of HGF on ES cell cardiogenesis were dramatically enhanced with the addition of 50 ng/ml bFGF. Under these conditions, 10/15 aggregates exposed to 50 ng/ml HGF displayed contractile activity that was initially detected in tiny regions throughout the culture by Induction Day 6. Regions of beating cells within a single aggregate were never synchronous; however, from Days 6-12, they continually expanded in size and could easily be identified, via immunostaining, as networks of cardiomyocytes that contained sarcomeres. Immunostaining also revealed that the majority of cells (>86% of total cells) in 100% of HGF-bFGF treated cultures were cardiogenic, displaying the characteristics of cells at all three stages of sarcomere assembly. Based on these results, it is concluded that HGF is a potent inducer of ES cell cardiogenesis, the effects of which are enhanced by bFGF.

Cardiac Induction of Mesoderm Progenitor Cells via HGF+EGF

EGF alone was modestly cardiogenic in the range of concentrations utilized for this study. Beating within any given induced aggregate was also confined to small foci of cells that correspondingly stained with muscle-specific proteins. It is interesting to note, however, that the percentage of EGF-treated aggregates with cardiogenic regions identified via immunostaining was usually equivalent to the percentage of aggregates that exhibited contractile activity (i.e., in most cases, if an aggregate did not beat, it also did not stain). This is in contrast to observations made with other growth factors whereby the extent of cardiac induction could not be accurately evaluated by observations of beating alone. In addition, the induction potential of EGF was minimally enhanced with bFGF supplementation. Taken together, these results suggest that EGF enhances cardiomyocyte maturation.

Identification of ES cell-Derived Cardiomyocytes via Immunohistochemistry

Cultures were fixed in 4% formaldehyde/phosphate-buffered saline for 45 minutes and permeabilized with 0.1% Triton X-100 for 45 minutes at 4° C. To minimize nonspecific binding of antibodies, cultures were preincubated in blocking buffer consisting of 2% donkey serum/1% BSA in phosphate-buffered saline for 1 hour at 4° C.

To assess cardiac induction, induced aggregates were incubated with monoclonal antibodies specific for sarcomeric myosin heavy chain (Developmental Studies Hybridoma Bank Antibody MF-20, Iowa City, Iowa) diluted 1:10 in blocking buffer. Alternatively, staining was performed with monoclonal antibodies specific to sarcomeric actin (1:400, Sigma). The secondary antibody was FITC-conjugated goat antimouse (1:400; ICN Pharmaceuticals, Inc, Aurora, Ohio). Antibody incubations were performed at 4° C. overnight in a humidified chamber, followed by extensive washing with phosphate-buffered saline. Cells were mounted in Vectashield Mounting medium (Vector Laboratories, Burlingame, Calif.) and observed on a Nikon Eclipse TE 300 microscope.

To enumerate the percentage of cardiac myocytes induced by mesoderm conditioned medium, induced and noninduced (control) aggregates were immunostained with antimyosin heavy chain and counterstained with propidium iodide (PI) (Molecular Probes, Eugene, Oreg.). Using a Leica TCS SP2 confocal microscope, ×100 fields in each EB were randomly selected for digital photomicrography. Based on myosin heavy chain expression, all PI-stained cells in each ×100 field were scored as a myocyte or nonmyocyte; by convention, red (PI) nuclei that were clearly surrounded by green (myosin heavy chain) staining were scored as myocytes. Between 1300 and 2000 cells were counted in each aggregate. For each condition, the average percentage (±SEM) of myocytes in 5 aggregates was determined; statistical significance between means was assessed using a 2-tailed t test with unequal variance. The percentage of myocytes was verified by disaggregating and replating the cells at nonconfluent density before immunohistochemical staining and counting.

Endothelium Induction of Mesoderm Progenitor Cells via BMPs

The cardiogenic efficacy of BMP-4 was examined. To this end, pluripotent ES cells were initially cultured in accordance with the methods described herein, and exposed to BMP-4 at various concentrations during the induction step. BMP-4 (50 ng/ml) in combination with HGF and FGF did not minimize the cardiogenic effects of HGF-FGF alone to any extent. However, when applied in the absence of HGF-FGF, relatively pure cultures of cardiomyocytes induced by the latter were dramatically and consistently (i.e., 100% of BMP-4 induced cultures) replaced by relatively pure populations of endothelial and smooth muscle cells that rapidly matured into three-dimensional vascular tubes.

Differentiation in Control Cultures is Specific to Mesoderm.

During the course of this investigation, control conditions were established that included the expansion and suspension of stem cells in accordance with the methods described above. Control cells were also subsequently plated onto a fibronectin substrate but allowed to differentiate in the absence of additional MesA factor supplementation. To this end, it was interesting to observe that control conditions consistently resulted in the differentiation of a limited number of mesoderm-derived cell types in the absence of detectable endoderm or ectoderm derivatives. These cell types included varying numbers of small cardiogenic foci, endothelial and smooth muscle cells that occasionally matured into vascular tube-like structures (see BMP-4), skeletal myotubes and a currently unidentified fibroblast-like cell population that often occupied peripheral regions within HGF-FGF-induced cardiogenic cultures (i.e., whereas approximately 86% of HGF-FGF induced cells were cardiomyocytes, a fibroblast-like cell type accounted for the remaining 10-14% of total cells). This is in contrast to the extended variety of differentiated cells types from all three germ layers typically observed under standard culture conditions, and provides strong support for the current claim to a unique induction system that directs ES cells into a mesodermal lineage.

The scope of the invention is not limited to the specific embodiments described herein. Rather, the claim should be looked to in order to judge the full scope of the invention.

What is claimed is:

1. An in vitro method of directly differentiating endothelial cells from human pluripotent cells, the method comprising:
expanding the pluripotent cells in the presence of a mesodermal priming medium;
suspending the expanded cells in the priming medium for a predetermined time period resulting in the formation of mesoderm aggregates, wherein mesoderm aggregates comprise a relatively pure population of mesoderm progenitor cells; and
exposing the mesoderm progenitor cells to a MesA factor that promotes endothelial cell differentiation.

2. The method of claim 1, wherein the mesodermal priming medium is serum free.

3. The method of claim 2, wherein the mesodermal priming medium further comprises a serum replacement.

4. The method of claim 1, wherein the mesodermal priming medium comprises Fibroblast Growth Factor (FGF).

5. The method of claim 4, wherein the Fibroblast Growth Factor is basic Fibroblast Growth Factor (bFGF).

6. The method of claim 1, wherein the mesodermal priming medium comprises a Bone Morphogenetic Protein (BMP) inhibitor.

7. The method of claim 1, wherein the mesodermal priming medium comprises an inhibitor of BMP activity.

8. The method of claim 1, wherein the mesodermal priming medium comprises a mesoderm inducing factor.

9. The method of claim 8, wherein the inducing factor is Fibroblast Growth Factor.

10. The method of claim 8, wherein the mesodermal priming medium further comprises a factor that inhibits non-mesoderm differentiation.

11. The method of claim 10, wherein the factor is Fibroblast Growth Factor.

12. The method of claim 9, wherein the Fibroblast Growth Factor is basic Fibroblast Growth Factor.

13. The method of claim 11, wherein the Fibroblast Growth Factor is basic Fibroblast Growth Factor.

14. The method of claim 10, wherein the factor is a Bone Morphogenetic Protein inhibitor.

15. The method of claim 10, wherein the factor inhibits Bone Morphogenetic Protein activity.

16. The method of claim 1, wherein the mesodermal priming medium further comprises fibroblast conditioned medium.

17. The method of claim 1, wherein the suspending and exposing steps are essentially free of feeder cells.

18. The method of claim 1, wherein the predetermined time is at least one day.

19. The method of claim 1, wherein the predetermined time is 3 days.

20. The method of claim 1, wherein the formation of mesoderm aggregates comprises a step that promotes cell-cell contacts between the expanded cells, wherein the cell-cell contacts promote mesoderm induction and endothelial cell differentiation.

21. The method of claim 1, wherein the mesoderm progenitor cells have the potential to differentiate to cardiomyocytes, endothelial cells, smooth muscle cells, mesodermal mesenchyme, hematopoietic cells, skeletal muscle cells, adipocytes, chondrocytes, osteocytes, and other mesoderm-derived cell types.

22. The method of claim 1, wherein the MesA factor comprises a Bone Morphogenetic Protein.

23. The method of claim 22, wherein the Bone Morphogenetic Protein is Bone Morphogenetic Protein-4 (BMP-4).

24. The method of claim 23, wherein the MesA factor promotes vasculogenesis.

25. The method of claim 22, wherein the MesA factor further induces at least one additional mesoderm derived cell type.

26. The method of claim 25, wherein the cell type comprises smooth muscle cells.

27. The method of claim 23, wherein the MesA factor further induces at least one additional mesoderm derived cell type.

28. The method of claim 27, wherein the cell type comprises smooth muscle cells.

29. The method of claim 1, wherein the efficacy of a potential MesA factor to promote the endothelial cell differentiation is experimentally determined and optimized by exposing the mesoderm progenitor cells to varying concentrations of the potential MesA factor and assessing the outcome.

30. The method of claim 1, wherein the efficacy of the MesA factor is modulated by the concentration of the MesA factor.

31. The method of claim 1, wherein the efficacy of the MesA factor is modulated by exposing the mesoderm progenitor cells to at least one additional MesA factor.

32. An in vitro method of directly differentiating endothelial cells from human pluripotent stem cells, the method comprising:
inducing the pluripotent cells into a relatively pure population of mesoderm progenitor cells; and
exposing the mesoderm progenitor cells to a MesA factor that promotes endothelial cell differentiation.

33. The method of claim 32, wherein the inducing step comprises the steps of:
expanding and priming the pluripotent stem cells in culture medium comprising a mesoderm inducing factor; and
suspending the primed cells in culture medium comprising a mesoderm inducing factor.

34. The method of claim 33, wherein the mesoderm inducing factor comprises Fibroblast Growth Factor (FGF).

35. The method of claim 34, wherein the Fibroblast Growth Factor is basic Fibroblast Growth Factor.

36. The method of claim 33, wherein the culture medium comprises a Bone Morphogenetic Protein (BMP) inhibitor.

37. The method of claim 33, wherein the culture medium comprises an inhibitor of Bone Morphogenetic Protein activity.

38. The method of claim 33, wherein the mesoderm inducing factor inhibits non-mesoderm differentiation.

39. The method of claim 33, wherein the culture medium is serum free.

40. The method of claim 39, wherein the culture medium further comprises a serum replacement.

41. The method of claim 33, wherein the inducing step further comprises exposing the primed cells to a compound that promotes the commitment and survival of the mesoderm progenitor cells.

42. The method of claim 41, wherein the compound comprises a component of extracellular matrix.

43. The method of claim 41, wherein the compound comprises fibronectin.

44. The method of claim 33, wherein the suspending step is essentially free of feeder cells.

45. The method of claim 33, wherein the culture medium further comprises fibroblast conditioned medium.

46. The method of claim 33, wherein the inducing step further comprises culture conditions that promote cell-cell contacts between the primed cells, wherein the cell-cell contacts promote mesoderm induction and endothelial cell differentiation.

47. The method of claim 33, wherein the inducing step further comprises the formation of aggregates, wherein the formation of aggregates promotes mesoderm induction and endothelial cell differentiation.

48. The method of claim 32, wherein the mesoderm progenitor cells have the potential to differentiate to cardiomyocytes, endothelial cells, smooth muscle cells, mesodermal mesenchyme, hematopoietic cells, skeletal muscle cells, adipocytes, chondrocytes, osteocytes, and other mesoderm-derived cell types.

49. The method of claim 32, wherein the MesA factor comprises a Bone Morphogenetic Protein.

50. The method of claim 49, wherein the Bone Morphogenetic Protein is Bone Morphogenetic Protein-4.

51. The method of claim 50, wherein the MesA factor promotes vasculogenesis.

52. The method of claim 49, wherein the MesA factor further induces at least one additional mesoderm derived cell type.

53. The method of claim 52, wherein the cell type comprises smooth muscle cells.

54. The method of claim 50, wherein the MesA factor further induces at least one additional mesoderm derived cell type.

55. The method of claim 54, wherein the cell type comprises smooth muscle cells.

56. The method of claim 32, wherein the efficacy of a potential MesA factor to promote endothelial cell differentiation is experimentally determined and optimized by exposing the mesoderm progenitor cells to varying concentrations of the potential MesA factor and assessing the outcome.

57. The method of claim 32, wherein the efficacy of the MesA factor is modulated by the concentration of the MesA factor.

58. The method of claim 32, wherein the efficacy of the MesA factor is modulated by exposing the mesoderm progenitor cells to at least one additional MesA factor.
